(19) United States
(12) Patent Application Publication
(10) Pub. No.: US 2007/0213596 A1
(43) Pub. Date: Sep. 13, 2007

(54) Minimal incision maximal access spine surgery instruments and method

(76) Inventor: James S. Hamada, Torrance, CA (US)

Correspondence Address: Curtis L. Harrington;Suite 250, 6300 State University Drive, Long Beach, CA, 90815, US

(21) Appl. No.: 11/374,311

(22) Filed: Feb. 7, 2006

(51) Int. Cl. A61B 1/32

(52) U.S. Cl. 600/219

(57) ABSTRACT

The system and method provides a retractor and use which gives the traditional ability to apply force and hold the surgical field open, but with the ability to select the depth, and shape of retraction tips for use during surgery, and in particular, spinal surgery. Any surgery may be carried out in an open surgical field with minimal patient trauma. A keyhole surgical opening accords a surgeon a maximum open operating field and necessitates only a minimal incision. An instrumentation-interchangeable tip junction facilitates the inserted support of interchangeable tips and may preferably include a connection shape which provides for a rotation lock.

### FIELD OF THE INVENTION

The present invention relates to improvements in the field of surgical methods and instrumentation. More specifically, the invention relates to methods and instruments for spinal surgery which enable relatively deep tissue retraction. Most specifically, the invention relates to a surgical retractor having a plurality of interchangeable members which will enable the surgeon to select the proper shape and depth for the exact structures which need to be retracted at any point in the surgery. Direct visualization may be customized at each and every point in the surgery to reduce error and maximize efficiency and safety of the procedure.

### BACKGROUND OF THE INVENTION

Conventional cross member retractors are known which have been fitted with flat retractor members for insertion into a surgical field, providing the surgical field is large enough to accommodate the width of the flat retractor members. Conventional, relatively wide retractor members can produce a significant arc sweep during use. As a result, if the surgeon is working with an opening which can accommodate the retractor blades, and if the surgical field is of the proper depth and orientation to accommodate the sweeping motion, conventional scissor-type instrumentation can be used. Such instrumentation is overly invasive and minimally customizable to the unexpected needs which may present during the surgical procedure. The complications due to the mismatch between instrumentation and needs are well documented and include patient harm, longer recovery periods and additional healing from excess tissue displacement to complete the surgical procedure.

Further, since the above described retractor is provided and usually utilized as an additional retractor to a main retraction system, it is likely that specialized circumstances are expected to invoke the need for supplemental retraction. However, having a supplemental retractor with the limitations of the above-described conventional retractor makes additional retraction capability only partially and narrowly marginally useful.

In most surgical procedures, a fine degree of manipulation on the part of the surgeon is required. Often, the lack of ability for fine, subtle manipulation will result in a less than complete action in the surgical field, or the possibility of damage where manipulations are attempted with instrumentation ill suited to such subtle manipulation. Unfortunately, the level and orientation of the subtlety required may unexpectedly change throughout the surgical procedure.

### SUMMARY OF THE INVENTION

The system and method of the invention provides a retractor having the traditional ability to apply force and hold the surgical field open, but with the ability to select the depth, and shape of retraction tips for use during surgery, and in particular, spinal surgery. Any surgery may be carried out in an open surgical field with minimal patient trauma. The method and apparatus of the present invention employs a keyhole surgical opening which accords a surgeon a maximum open operating field and necessitates only a minimal incision. The open procedure allows direct visualization of the surgical field, either with the naked eye or through the use of optical devices such as surgical microscopes and/or loupes the like, and thereby eliminates the need to employ devices such as endoscopes. The keyhole surgical opening permits a surgeon to utilize normal binocular vision and provides ready access for surgical tools. A section above the point of connection of the interchangeable tips are diverted slightly outwardly to insure that the tips and their connector move out of visual alignment into the surgical field.

In a most preferred embodiment, the members which extend into the surgical field angle apart from each other and then angle back into a parallel relationship before terminating at a connection end. This angled “jog” outward structure will be positioned below, but near the opening of the surgical field and can facilitate the co-location of supplementary structures ranging from suction to fiber optic illumination.

The connection end can include insertion into a support collar on the interchangeable tips or the support collar can be located on the instrumentation side of the instrumentation-interchangeable tip junction. The inserted support of the interchangeable tips will preferably include a connection shape which provides for a rotation lock. The rotation lock can provide for a single rotational position or multiple positions. A simple, one-position rotational lock can be utilized for interchangeable retractors not expected to assume more than one position, while a multi-position lock can be utilized for more subtle applications. As by example, where the mechanical distance between the point of pivot and opening of the surgical field is short, the rotation to achieve a given sized opening will be more significant. Providing multiple, slightly changing angled connection will enable opening of the retractor to create a more bilaterally symmetrical position of the retractor blades.

### BRIEF DESCRIPTION OF THE DRAWINGS

The invention, its configuration, construction, and operation will be best further described in the following detailed description, taken in conjunction with the accompanying drawings in which:

FIG. 1 is a perspective view of a retractor in accord with the present invention;

FIG. 2 is a sectional view taken along lines 2-2 of a first terminal portion of the distal part of the main portion of the retractor of FIG. 1 with respect to an interchangeable retractor tip seen in FIG. 1 and illustrating one possible interconnection geometry;

FIG. 3 is a sectional view taken along lines 3-3 as in FIG. 2 but shown in mated position;

FIG. 4 is a horizontal section taken along line 3-3 and illustrating one possible arrangement for a one-position rotational locking of the interchangeable retractor tip with respect to the distal part of the main portion of the retractor of FIG. 1;

FIG. 5 is a closeup section of a first angled portion which may facilitate a utility opening for fiber optic light cable, suction, and the like;

FIG. 6 is a plan view of the angular displacement operation of the retractor system;

FIG. 7 is a plan view of front of the retractor system seen in FIG. 1 which emphasizes the double curvature for increased lateral displacement of the interchangeable tips of the retractor system;

FIG. 7 is a plan view of front of the retractor system seen in FIG. 1 which emphasizes the double curvature for increased lateral displacement of the interchangeable tips of the retractor system;

FIG. 8 illustrates a plan view of an alternative shape for a front of the retractor system seen in FIG. 1 which is completely linear for absolute minimum lateral displacement upon insertion into the surgical field;

FIG. 9 illustrates a view of a first interchangeable tip seen with the tipmost end of the outwardly curved portion extending out of the page;

FIG. 10 illustrates an interchangeable tip having a curved retractor tip but having an overall length shorter than that shown in FIG. 9;

FIG. 11 illustrates an interchangeable tip having a curved retractor tip but having an overall length shorter than that shown in FIGS. 9 and 10;

FIG. 12 illustrates a bottom view of the interchangeable retractor tips seen in FIGS. 9-11;

FIG. 13 illustrates an interchangeable bladed retractor member;

FIG. 14 illustrates an interchangeable bladed retractor with a supported blade member having a length shorter than the supported blade member seen in FIG. 13;

FIG. 15 illustrates an interchangeable bladed retractor with a supported blade member having a length shorter than the supported blade member seen in FIGS. 13 and 14;

FIG. 16 illustrates a bottom view of the supported blade member interchangeable retractor tips seen in FIGS. 13-15;

FIG. 17 illustrates an exploded view of a more complex mounting system for the interchangeable tips;

FIG. 18 is a cross sectional rendering of the components seen in FIG. 17;

FIG. 18 illustrates a cross sectional view of the joined components seen in FIGS. 17-19;

FIG. 20 is a view taken along line 20-20 of FIG. 17 and illustrating a bottom view of an orientational adapter;

FIG. 21 is a view taken along line 21-21 of FIG. 17 and illustrating a top view of a threaded body of an interchangeable retractor tip;

FIG. 22 is a lower plan view of a retractor assembly which includes both a curved tip and which supports a rectangular blade;

FIG. 23 is a side view of the retractor assembly seen in FIG. 22;

FIG. 24 is a side view of the retractor assembly as seen in FIG. 23, but illustrating an embodiment having a series of lateral cuts;

FIG. 25 is a side view of a flexible curved tip retractor;

FIG. 26 is a view as seen in FIG. 25 but illustrating the flexing action when force is applied to the curved portion;

FIG. 27 is a lower side view of a retractor assembly which includes both a curved tip and which supports a shaped blade having an expanded circular lower area;

FIG. 28 is a plan view of the retractor assembly seen in FIG. 27;

FIG. 29 is a plan view of an embodiment similar to the retractor assembly seen in FIGS. 27 and 28, but where a support post is cylindrical and stops short of extending to the lower edge of the expanded circular lower area;

FIG. 30 is a lower side view of a retractor assembly which includes both a curved tip and which supports a shaped blade having an expanded pointed rectangular head lower area;

FIG. 31 is a plan view of the retractor assembly seen in FIG. 30; and

FIG. 32 is a plan view of an embodiment similar to the retractor assembly seen in FIGS. 27 and 28, but where a support post is cylindrical and stops short of extending to the lower edge of the expanded pointed rectangular head lower area.

### DETAILED DESCRIPTION OF THE PREFERRED EMBODIMENT

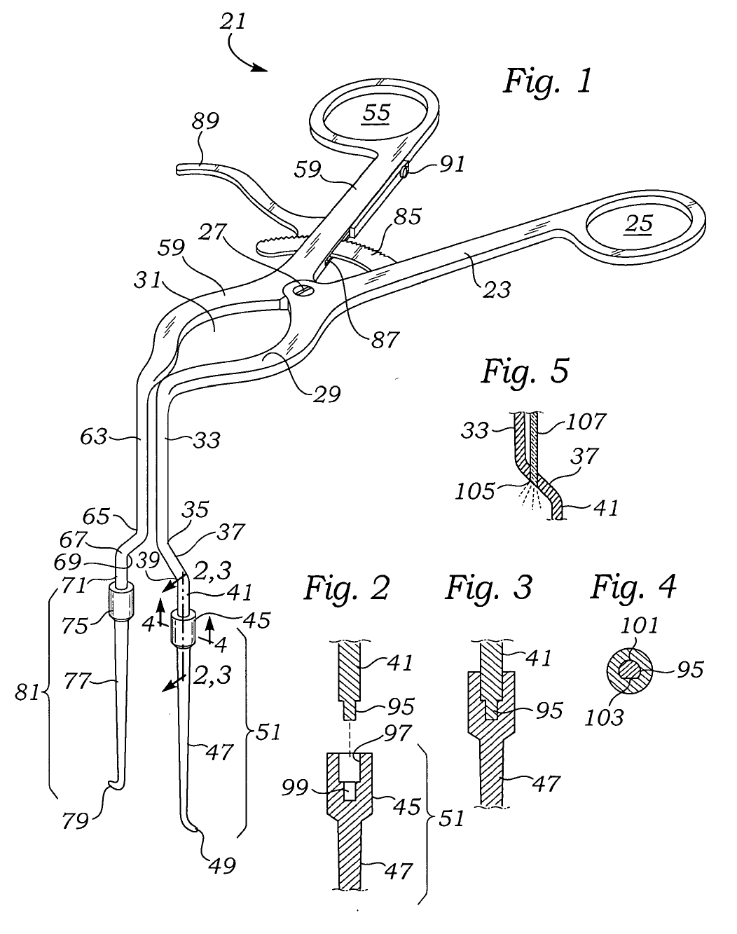

The description and operation of the retractor system will be best described beginning with reference to FIG. 1 and referred to as a retractor system 21. Retractor system 21 is particularly useful in providing a keyhole access opening. Retractor system 21 bears some general similarity to retractors generally known as Williams retractors, Gelpi retractors, Velpi retractors, Caspar retractors, or Ducker retractors; but the retractor system 21 differs therefrom with regard to some significant and important details.

Retractor system 21 includes a first member 23 having a loop opening 25 and a pivot point 27. Pivot point 27 is depicted as being a screw, but any method of providing stable pivot is an equivalent structure. Beyond the pivot point 27, the first member is continuous with a curved member 29 which provides an outward curvature to create a central space 31. Central space 31 provides a visual opening even when the retractor system 21 is closed. As will be seen, upon actuation of the retractor system 21, the curved member 29 will help provide additional clearance around the surgical field.

Curved member 29 is continuous with a first main retraction portion 33 which is generally at a right angle with respect to the main planar extent of the curved member 29 and is oriented to extend through a surgical field opening. At the end of the first main retraction portion 33, the first main retraction portion 33 angles outward at a first bend 35 and into a first angled portion 37. The first angled portion 37 extends through a second bend 39, and then into a first terminal portion 41. First terminal portion 41 is shown as leading into a first force fitting 45. From the first force fitting 45, a first interchangeable curved retractor body 47 extends. The first interchangeable curved retractor body 47 is seen as having an outwardly curved portion 49.

It is understood that the first force fitting 45 can be affixed to, continuous with or otherwise associated with either the first interchangeable curved retractor body 47 or the first terminal portion 41. In the figures which immediately follow, the first force fitting 45 will be shown as associated with the first interchangeable curved retractor body 47. Either orientation may have associated advantages. The system in which the first force fitting 45 is associated with the first interchangeable curved retractor body 47 will enable interchange of tips without the need to invert the retractor system 21. The first interchangeable curved retractor body 47, and first force fitting 45 may be referred to as a first interchangeable tip 51.

The other main structural portion of the retractor system 21 generally mirror those just described. A second member 53 having a loop opening 55 and shares the pivot point 27, with the point 27 on second member 53 being underneath a portion of first member 23 which overlies it. Beyond the pivot point 27, the second member 53 is continuous with a curved member 59 which provides a symmetric outward curvature to curved member 29.

Curved member 59 is continuous with a second main retraction portion 63 which is generally at a right angle with respect to the main planar extent of the curved member 59 which is generally at a right angle with respect to the main planar extent of the curved member 29 and is oriented to extend through a surgical field opening. Further, second main retraction portion 63 is generally parallel to first main retraction portion 33 and has the ability to move into close parallel relationship with first main retraction portion 33.

At the end of the second main retraction portion 63, the second main retraction portion 63 angles outward at a third bend 65 and into a second angled portion 67. The second angled portion 67 extends through a fourth bend 69, and then into a second terminal portion 71. Second terminal portion 41 is shown as leading into a second force fitting 75, then to a second interchangeable curved retractor body 77 having an outwardly curved portion 79. The second interchangeable curved retractor body 77, and second force fitting 75 may be referred to as a second interchangeable tip 81.

Between the two main structural portions of the retractor system 10, a number of different methods of holding the opening force may be applied. In the design shown, a conventional locking mechanism for enabling user selectably in applying displacement, opening force and immobilization of the position and holding force without having to continuously manually engage the retractor system 10.

The mechanism shown includes a toothed locking bar 85 which projects from the first member 23. The toothed locking bar 85 passes through an opening 87 in the second member 53 and is engageable by a locking lever 89 affixed to pivot with respect to the second member 53 into and out of engagement with the toothed locking bar 85. A spring 91 is shown positioned to urge the locking lever 89 into its positive engage position with respect to the toothed locking bar 85, and against which physical release force of the locking lever 89 will occur.

Referring to FIG. 2, one possible configuration for the interfitting of either the first and second terminal portions 41 and 71 is shown. Only first terminal portion 41 and first force fitting 45 will be illustrated, but the mechanism for these structures is expected to be identical and symmetric for the structures employed for the second terminal portion 71 and second force fitting 75. Also, in FIG. 2, the first force fitting 45 is shown as continuous and associated with the first interchangeable curved retractor body 47, but this need not be the case. Further, force fitting structure can, in different designs, be shared between the first interchangeable curved retractor body 47 and the first terminal portion 41.

First terminal portion 41 is seen to end with the provision of a reduced width portion 95. It is to be understood that the reduced width portion 95 need not be round, and can be triangular, square, pentagonal, hexagonal, heptagonal or polygonal. The first terminal portion 41 and reduced width portion 95 can be such that, taking for example the first interchangeable tip 51 with its outwardly curved portion 49, the first interchangeable tip 51 may assume a number of pre-specified orientations. For example, a reduced width portion which is triangular shaped can enable first interchangeable tip 51 to assume three orientations and a reduced width portion which is hexagonal shaped can enable first interchangeable tip 51 to assume six orientations. Other configurations can include a key projection which can interfit into 1, 2, 3 or more matching slots, where the matching slots are of defined angular orientation.

FIG. 2 also illustrates a stepped blind bore within the first force fitting 45 and which includes a first bore 97 for fittably engaging a portion of the first terminal portion 41 just above the reduced width portion 95. A second longitudinally deeper and narrower bore 99 interfits with the reduced width portion 95. The bores 97 and 99 need not be circular, they can be of any shape, any cylindrical shape, smooth or not. Further, depending upon materials, the first force fitting 45 may have been shown in an overly exaggerated width. It is shown to emphasize that depending upon the materials selected, that some additional lateral material may be necessary to provide good hold and fit with respect to the structures including the reduced width portion 95 and optionally the portion of the first terminal portion 41 above it.

Further, depending upon selection of materials, the first bore 97 might be eliminated and it is possible that the only bore within the first force fitting 45, if such a first force fitting 45 is needed at all, might be a single bore seen as the second longitudinally deeper and narrower bore 99.

Further, other features can be employed to insure that the first interchangeable tip 51 remains vertically engaged with the first terminal portion 41, regardless of the mating structures. Mechanical features can include a locking detent, or interference fit, or slight reverse interfering taper. Where the retractor system is made of ferromagnetic material, one or both of the first interchangeable tip 51 and first terminal portion 41 can be made of a magnetic material such that the first interchangeable tip 51 is magnetically attracted to and held by the first terminal portion 41. Where other ferromagnetic materials or sensors may be present within the surgical field, it may be advantageous that only the reduced width portion 95 be magnetized, or perhaps where only the material of the first force fitting 45 at the face end of the second longitudinally deeper and narrower bore 99. In another embodiment, both the second longitudinally deeper and narrower bore 99 and the reduced width portion 95 may be cylindrical and threaded. Many other possible joinder structures may have advantage in joining the first interchangeable tip 51 to the first terminal portion 41. It should be remembered that the main force to be applied to this junction will be lateral and that the axial force of holding need not be great.

Referring to FIG. 3, the first interchangeable tip 51 is shown as closely interfitting with and being supported by the first terminal portion 41. It must be emphasized that the members seen in FIG. 2 can be of any shape or orientation.

Referring to FIG. 4, a horizontal section taken along line 3-3 of FIG. 1 illustrates one of thousands of possible orientations and shapes for the first interchangeable tip 51 second longitudinally deeper and narrower bore 99 and first terminal portion 41's reduced width portion 95 seen in FIGS. 2 and 3. Here, the reduced width portion 95 is seen as having a shape which includes a cylindrical diameter 101 and a flat portion 103 which mates with the complementary structures within the second longitudinally deeper and narrower bore 99. One advantage of this particular orientation is that the shape for the first interchangeable tip 51 readily admits the reduced width portion 95 into the first bore, and then facilitates the location of the reduced width portion 95 into its matching shaped second longitudinally deeper and narrower bore 99 by turning until the flat portion 103 of the reduced width portion 95 is able to align with a complementary feature of second longitudinally deeper and narrower bore 99. Thus the interfitting structures do not force the surgical practitioner to pull his attention away from the surgical field and take time and excess dexterity to interfit more demanding interlocking structures.

Referring to FIG. 5, a closeup view of the first angled portion 37 located between the first main retraction portion 33 and first terminal portion 41 illustrates the possibility of forming a through bore 105 to facilitate passage of a utility conduit 107. The utility conduit shown is a fiber cable 107. Both the location and lower surface presented by first angled portion 37 permit easy access lighting of the tissues surrounding first and second interchangeable curved retractor tips 47 and 77. This location will also likely not be optically obscured by foreign object matter or liquids as any liquids would tend to gravitationally run away from the presented end of fiber optic cable 107. Other components can be utilized in place of the utility conduit shown, including suction, sensors, and the like.

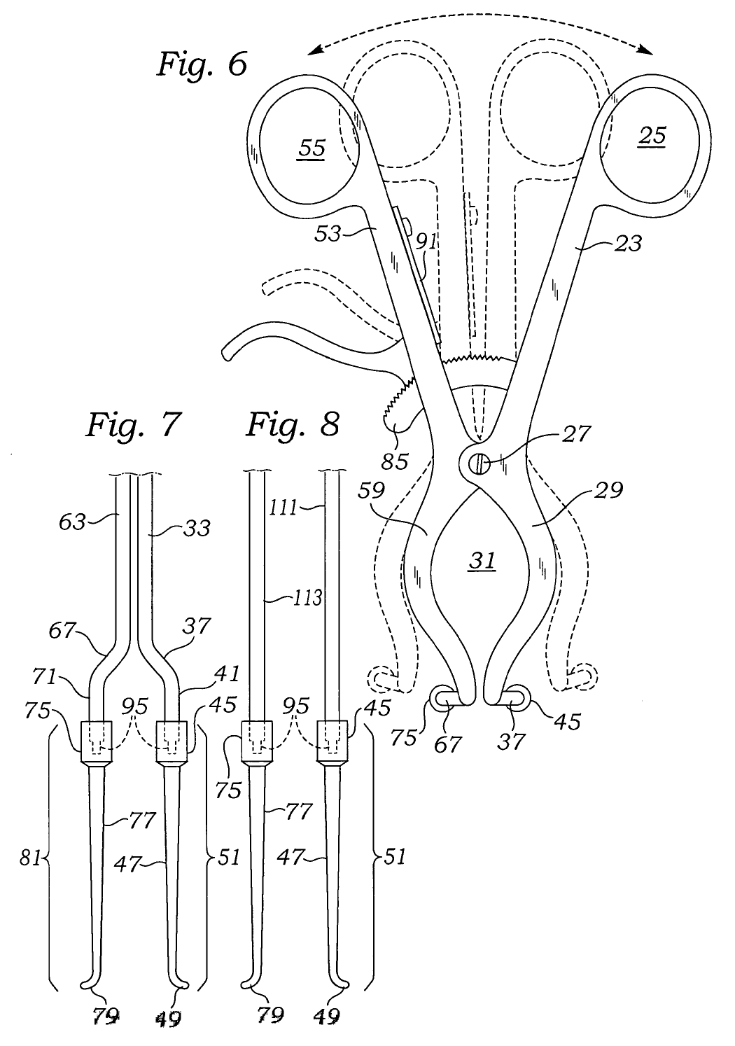

Referring to FIG. 6, a top view illustrating the action of movement of the retractor system 21 is shown. The ratchet enables the first and second members 23 and 53 to come together to the dashed line position while creating an enlarged central space 31, and without the need for manual actuation of the locking lever 89, which is urged into engagement with the toothed locking bar 85 by spring 91.

Referring to FIG. 7 a plan view of the retractor system 21 seen in FIG. 1 is seen from the perspective of a position in front of the retractor system 21. This view shows the extent of outward curvature of the outwardly curved portions 49 and 79, and illustrates the relative lengths of the first and second terminal portions 41 and 71 with respect to the lengths of the first and second interchangeable tips 51 and 81.

Referring to FIG. 8 a portion of a second embodiment is shown in which the first and second main retraction portions 33 and 63, first and second angled portions 37 and 67, and the first and second terminal portions 41 and 71 are replaced by completely straight first and second main retraction portions 111 and 113 which include the reduced width portion 95 at their terminal ends. As can be seen, the first and second interchangeable tips 51 and 81 are engaged in the same manner as was seen in FIGS. 1-4. It is again understood, however, that the method for interconnection detailed in FIGS. 2 and 3, and particularly FIG. 4 are only representative of one method of hundreds by which interconnected support of the first and second interchangeable tips 51 and 81 can be accomplished.

The combination of the first and second interchangeable tips 51 and 81 with a retractor system 21 having straight first and second main retraction portions 111 and 113 will enable initial entry into a smaller size keyhole opening. As a result, it may be preferable to begin with retractor system 21 having straight first and second main retraction portions 111 and 113 to then be replaced with a retractor system 21 having first and second main retraction portions 33 and 63, first and second angled portions 37 and 67, and the first and second terminal portions 41 and 71, in order to provide a surgical field with greater clearance. The views of FIGS. 7 and 8 illustrate that for a given sized exterior patient surgical opening, a retractor system 21 with straight first and second main retraction portions 111 and 113 can fit but will be limited in retraction of the lower tissues by the surgical opening. FIGS. 7 and 8 illustrate that with straight first and second main retraction portions 111 and 113, the first and second interchangeable tips 51 and 81 can be brought more closely together to facilitate entry into a surgical opening. Some retraction of the opening can be accomplished to enable retractor system 21 having first and second main retraction portions 33 and 63, first and second angled portions 37 and 67, and the first and second terminal portions 41 and 71 so that the surgical field will have the structures, including first and second force fittings 45 and 75 further out of view in the surgical field.

With regard to the first and second interchangeable tips 51 and 71, a number of different lengths may be provided in order to maximally facilitate the different working depths the surgeon may encounter. As was the case with the initial use of a retractor system 21 having straight first and second main retraction portions 111 and 113 to then be replaced with a retractor system 21 having first and second main retraction portions 33 and 63, first and second angled portions 37 and 67, and first and second terminal portions 41 and 71, the practitioner can begin work in the surgical opening with shorter lengths of first and second interchangeable tip 51 and 81, and gradually change to longer lengths of first and second interchangeable tip 51 and 81 as the work progresses.

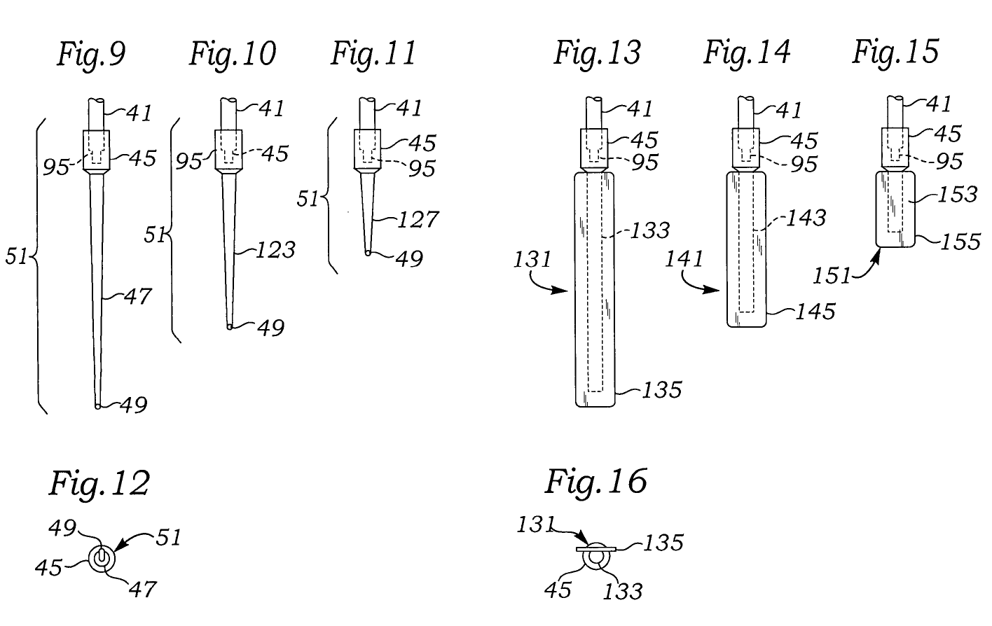

Referring to FIG. 9 a view of the first interchangeable tip 51 is seen with the tipmost end of the outwardly curved portion 49 facing the viewer. This the first interchangeable tip 51 is the same as has been seen in FIGS. 1-4, 7 & 8. Referring to FIG. 10, an interchangeable tip 121 having a curved retractor tip 123 having an overall length shorter than the curved retractor tip 47. The dimensions of the force fitting 45 are the same as for interchangeable tip 51. Although not referred to as a “first” or “second” interchangeable tip 121 or curved retractor tip 123, it should be emphasized that the first and second interchangeable tip 51 and 81 may be made identical or can be made differently.

Referring to FIG. 11 an interchangeable tip 125 having a curved retractor tip 127 having an overall length shorter than the curved retractor tip 123 is shown. FIGS. 9, 10, and 11 are but three length sizes of interchangeable tip 51, 121, and 125. Other variations are possible, including differing diameter and different shapes of the outwardly curved portion 49, as well as the absence or presence of outwardly curved portion 49.

Referring to FIG. 12, a bottom view of the interchangeable tip 51, which is equivalent to the interchangeable tip 51, 121, and 125 is shown. The bottom view seen in FIG. 12 is the “footprint” of the interchangeable tip 51, and will be seen with respect to the other embodiments to better illustrate the shapes thereof.

Referring to FIG. 13, an interchangeable bladed retractor member 131 is illustrated with respect to the first terminal portion 41 seen in previous figures. The upper portion of the interchangeable bladed retractor member 131 is identical to that seen for the first interchangeable tip 51, including the force fitting 45 and the reduced width portion 95 which interfits with the first bore 97 and second longitudinally deeper and narrower bore 99, respectively. Below the force fitting 45, a support post 133 supports a blade 135 on one side of the support post 133. The blade 135 is seen extending slightly below the support post 133 by about the width of the support post 133. Depending upon the method of attachment, the blade 135 can be attached to a cylindrical shaped support post 133 by surface welding, or in the alternative, the support post 133 can have a flattened area to support a weld with more surface area coverage.

For the given width seen in FIG. 13, a interchangeable bladed retractor member 131 can have various lengths. Referring to FIG. 14, an interchangeable bladed retractor member 141 is also illustrated with respect to the first terminal portion 41 seen in previous figures. Again, the upper portion of the interchangeable bladed retractor member 131 is identical to that seen for the first interchangeable tip 51. A slightly shorter support post 143 supports a blade 145 on one side of the support post 143, with the blade 145 is seen extending slightly below the support post 143 by about the width of the support post 143.

Similarly, referring to FIG. 15, an interchangeable bladed retractor member 151 is illustrated. A still further shortened support post 153 supports a blade 155 on one side of the support post 153, with the blade 155 is seen extending slightly below the support post 153 by about the width of the support post 153. Referring to FIG. 16, a bottom view of the interchangeable bladed retractor member 131 is equivalent to a bottom view for the interchangeable bladed retractor members 141 and 151. FIG. 16 illustrates that the support post 133 (and the support posts 143 and 153) include a flattened area to better support the flat side of the blade 135.

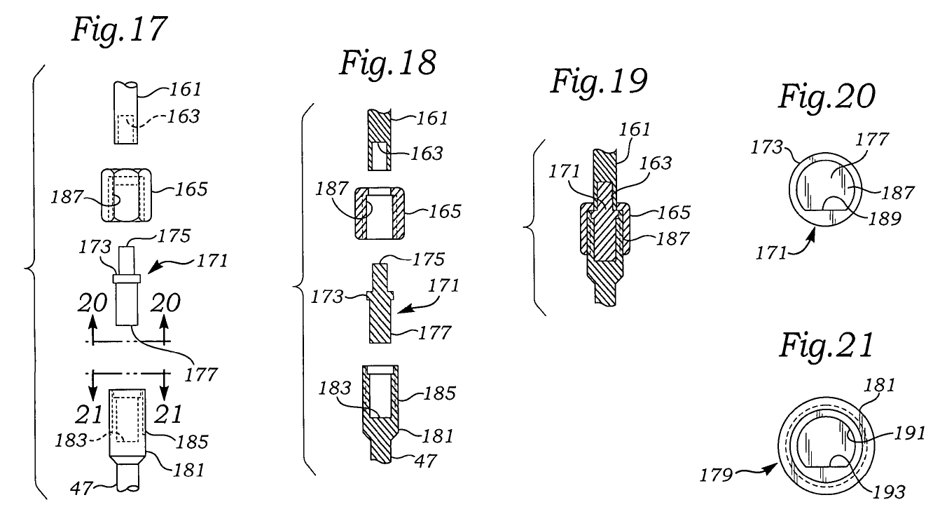

Referring to FIG. 17, a more complex mounting system for the interchangeable tips is illustrated. An terminal portion 161 which can be either a first or second terminal portion is seen as having first shaped bore 163, seen as opening downwardly. A locking nut 165 is configured to axially fit partially over at least the tip end of the terminal portion 161. Depending upon the design, the action of the locking nut can be circumferentially compressive or it can depend from a rim (not seen) on the bottom periphery of the terminal portion which may engaged with an upper shoulder of the locking nut 165.

Below the locking nut 165 is seen an orientational adapter 171. Orientational adapter 171 has an optional central land 173 which may for a separation between an upper projection 175 and a lower projection 177. The land 173 is optional because the bores into which the upper projection 175 and lower projection 177 will fit have defined dimensions and consequently the presence of a land is un-necessary. The land 173 is un-necessary even where the effective widths and lengths of the upper projection 175 and lower projection 177 are the same.

Below the orientational adapter 171, an interchangeable tip 179, which may be representative of any of the members 51, 81, 121, 125, 131, 141, and 151, is shown. Interchangeable tip 179 has a second, upwardly directed, threaded body bore 183 which complementary to the lower projection 177. Interchangeable tip 179 has a force fitting threaded body 181 also has an external thread set 185 which are complementary to an internal thread set 187 carried within the locking nut 165. Referring to FIG. 18, a sectional view of the components seen in FIG. 17 are shown. FIG. 19 is a sectional view illustrating the components seen in FIGS. 17 and 18 in an attached position.

Although the components seen in FIGS. 17-19 are more complex, the selection of the first shaped bore 163, upper projection 175 and lower projection 177, and threaded body bore 183 enables the user to select a different orientational adapter 171 to achieve a different rotational lockup. Where the shapes of the bores 163 and 183, along with their complementary projections 175 and 177, admit only to a single orientational lockup, different orientational adapters 171 can be employed to achieve different angular positions of the members 51, 81, 121, 125, 131, 141, and 151. As before, where polygonal shapes are used for the bores 163 and 183, along with their complementary projections 175 and 177, different orientations can be achieved with a single orientational adapter 171. However, the use of a geometry with a single rotational position invites the use of orientational adapters 171 in which the angle can be specifically set.

Referring to FIG. 20, a view taken along line 20-20 of FIG. 17 illustrates lower projection 177. The shape of lower projection 177 is seen as having a generally cylindrical surface 187 with a flat side 189. Referring to FIG. 21, a view taken along line 21-21 of FIG. 17 illustrates details of the threaded body bore 183 and the upper end of the external thread set 185. The shape of the threaded body bore 183 is seen as having a generally cylindrical surface 191 interrupted by a flat side 193.

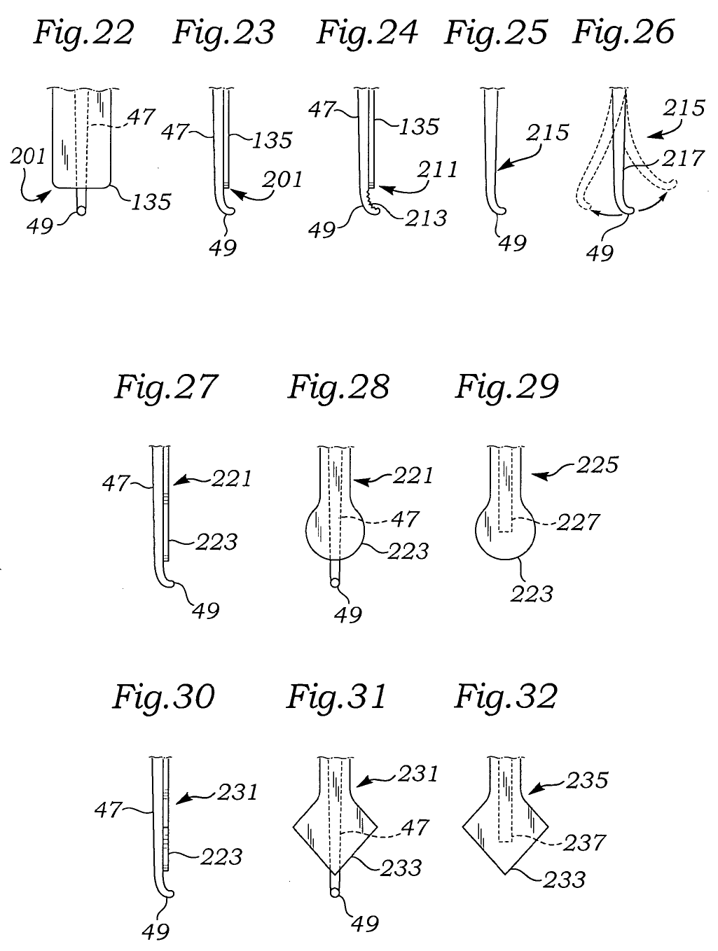

FIGS. 22-33 illustrate a number of different shapes which are possible for use as the interchangeable retractor tips which could be substituted for structures 47, 77, 127, 131, 143, and 151. All of the structures 47, 77, 127, 131, 143, and 151, as well as the structures seen in FIGS. 22-33 could be utilized in conjunction with any attachment system, including the systems outlined in FIGS. 17-19 and the system outlined in FIGS. 2-4. Referring to FIG. 22, a retractor 201 includes a curved retractor tip 47 structure having a curved portion 49 curving toward a blade 135 of a length extending short of the curved portion 49 is shown. Referring to FIG. 23, a side view of the retractor assembly 201 is shown and gives a better visual illustration of the orientation of the curved portion 49 and blade 135. In this configuration, the blade surface 135 can provide a relatively wider sweeping area and action while the slightly lower extending curved portion 49 of curved retractor tip 47 can provide a base gathering and collection action to insure that the area swept by the retractor assembly 201 will be clear.

Referring to FIG. 24, a side view illustrates a retractor assembly 211 which has the same overall construction as was seen for retractor assembly 201, except for the formation of a series of lateral cuts 213 in the upper surface of the curved portion 49. The lateral cuts 213 form a series of teeth like projections which help stabilize any tissue gathered onto the curved portion 49 and helps to prevent any gathered tissues from sliding off, around the tip of the curved portion 49. Avoiding slide off is particularly important in the moist surgical field.

Referring to FIG. 25 a retractor assembly 215 having a construction similar to curved retractor tip 45 is seen. The main length of the body 217 is made of a flexible material. The flexible material construction may extend throughout the body 217, or the body 217 may be differentially hardened. Referring to FIG. 26, the flexing of the body 217 is shown. Note that the curved portion 49 is not seen as straightening to any appreciable extent. This can be achieved naturally by providing more strength in the curved portion 49 or by the natural geometry which enables the body 217 to have the greatest flexure along the body 217 length. Body 217 can also have a taper to help control its flexing. The ability of the retractor assembly 215 to flex gives it the ability to avoid tissue damage at the maximum depth over which retraction occurs. The maximum depth is an area swept farthest from the surgical practitioner's view and the area most prone to contact with structures not already revealed at the surgical opening. This feature enables a gentler mode of retraction, especially where used with softer or more delicate tissues.

Referring to FIG. 27, a retractor assembly 221 includes a curved retractor tip 47 structure having a curved portion 49 curving toward a specially shaped blade 223 of a length extending short of the curved portion 49. Referring to FIG. 28, a side view of the retractor assembly 223 is shown and gives a better visual illustration of the shape of the blade 223. Blade 223 is in the shape of an inverted lollipop or keyhole shape which blends into a linear shape extending upward. This shape provides a wider retraction at a relatively lower depth inside the surgical field.

Referring to 29, a retractor 225 includes the shaped blade 223 seen in FIGS. 27 and 28, but in lieu of the curved retractor tip 47, a shortened support post 227 extends only to a point significantly short of the lowest extent of the specially shaped blade 223. This enables the lower shape of the retractor assembly 225 to have a shape and contour matching the specially shaped blade 223.

Referring to FIG. 30, a retractor assembly 231 includes a curved retractor tip 47 structure having a curved portion 49 curving toward a specially shaped blade 233 of a length extending short of the curved portion 49. Referring to FIG. 31, a side view of the retractor assembly 233 is shown and gives a better visual illustration of the shape of the blade 233. Blade 233 is in the shape of a diamond head or pointed square shape which blends into an upper linear portion. This shape provides a wider retraction at a relatively lower depth inside the surgical field, but the pointed lateral corners also provide a wider retraction slightly displaced from the lower corner or tip.

Referring to 32, a retractor 235 includes the shaped blade 233 seen in FIGS. 30 and 31, but in lieu of the curved retractor tip 47, a shortened support post 237 extends only to a point significantly short of the lowest extent of the specially shaped blade 223, seen below the lateral corners but above the lowermost corner. This enables the lower shape of the retractor assembly 225 to have a shape and contour matching the specially shaped blade 233.

While the present system has been described in terms of a minimal incision maximal access spine surgery instruments and method, and includes any method of actuation, despite being shown as being centrally discussed as a manually actuatable with a scissors-like device, one skilled in the art will realize that the structure and techniques of the present system can be applied to many appliances including any appliance which utilizes the several aspects of the embodiments of the instrumentation of the system or any process which utilizes the steps of the inventive system.

Although the system of the invention has been derived with reference to particular illustrative embodiments thereof, many changes and modifications of the system, retractors, and components may become apparent to those skilled in the art without departing from the spirit and scope of the inventive system. Therefore, included within the patent warranted hereon are all such changes and modifications as may reasonably and properly be included within the scope of this contribution to the art.

What is claimed:

1. An instrument comprising:
a first main retraction portion having a first end and a second end;
a first interchangeable retractor tip having a first end for removable connection to said second end of said first main retraction portion, and rotationally fixed with respect to said second end of said first main retraction portion, and a second end;
a second main retraction portion having a first end and a second end;
a second interchangeable retractor tip having a first end for removable connection to said second end of said first main retraction portion, and rotationally fixed with respect to said second end of said second main retraction portion, and a second end;
means, connected to said second ends of said first and second main retraction portions, to move said first main retraction portion away from said second main retraction portion to create a minimal access clear surgical field.

2. The instrument as recited in claim 1 and further comprising:
a first force fitting associated with one of said first main retraction portion and said first interchangeable retractor tip; and
a second force fitting associated with one of said second main retraction portion and said second interchangeable retractor tip.

3. The instrument as recited in claim 2 wherein said first force fitting is continuous with said first interchangeable retractor tip and includes a first force fitting first bore for interfitting with said first end of said first main retraction portion, and wherein said second force fitting is continuous with said second interchangeable retractor tip and includes a second force fitting first bore for interfitting with said first end of said second main retraction portion.

4. The instrument as recited in claim 3 wherein said first and second bores in said first and second force fittings are non-completely cylindrical shaped and said rotational fixation is achieved due to said non-completely cylindrical shape.

5. The instrument as recited in claim 3 wherein said first force fitting includes a first force fitting second bore having a depth greater than said first force fitting first bore, and wherein said second force fitting includes a second force fitting second bore having a depth greater than said second force fitting first bore, and wherein said first force fitting second bore and said second force fitting second bore is non-completely cylindrical shaped and said rotational fixation is achieved due to said non-completely cylindrical shape.

6. The instrument as recited in claim 1 wherein said first and said second interchangeable retractor tips each have a tapered body and including a curved portion near said second ends.

7. The instrument as recited in claim 6 wherein said first and said second interchangeable retractor tips each have a blade centrally attached to one side of said tapered body and including a curved portion near said second ends.

8. The instrument as recited in claim 7 wherein said blade has a rectangular shape.

9. The instrument as recited in claim 7 wherein said blade has a first end adjacent said curved portion which is circular shaped.

10. The instrument as recited in claim 7 wherein said blade has a first end adjacent said curved portion which has a pointed rectangular shape.

11. The instrument as recited in claim 1 wherein said first and said second interchangeable retractor tips each have a cylindrical body and including a blade near said second ends first and said second interchangeable retractor tips and which extends beyond said second ends of said first and said second interchangeable retractor tips.

12. The instrument as recited in claim 11 wherein said blade has a rectangular shape.

13. The instrument as recited in claim 11 wherein said blade has a first end adjacent said second end of said first and said second interchangeable retractor tips having a curved portion which is circular shaped.

14. The instrument as recited in claim 11 wherein said blade has a first end adjacent said second end of said first and said second interchangeable retractor tips having a curved portion which has a pointed rectangular shape.

15. The instrument as recited in claim 2 and wherein said first force fitting includes:
a first locking nut having an internal thread and an opening for fitting over said first end of said a first main retraction portion;
a first threaded body continuous with said first interchangeable retractor tip and includes a threaded body bore;
a first orientational adapter having a first end and a second end for fitting within said first threaded body bore, and wherein said first main retraction portion includes a first main retraction bore for interfitting with said first end of said first orientational adapter;
a second locking nut having an internal thread and an opening for fitting over said second end of said a second main retraction portion;
a second threaded body continuous with said second interchangeable retractor tip and includes a threaded body bore;
a second orientational adapter having a first end and a second end for fitting within said second threaded body bore, and wherein said second main retraction portion includes a second main retraction bore for interfitting with said first end of said second orientational adapter, and wherein the shapes of said first and second ends of said first and said second orientational adapters are used in conjunction with said threaded body bores of said first and said second threaded bodies and said first and second main retraction bores to rotationally fix a position of said first main retraction portion with respect to said first interchangeable retractor tip and to rotationally fix a position of said second main retraction portion with respect to said second interchangeable retractor tip.

16. The instrument as recited in claim 1 wherein said first and said second main retraction portions each have a first curve away from each other and then second curve into a parallel relationship to enable said first and second interchangeable retractor tips have a slight lateral displacement from each other to provide a less obstructed surgical field.

17. The instrument as recited in claim 1 wherein at least one of said first and said second main retraction portions has a traversing bore between said first and said second curves for accommodating a utility conduit.

18. The instrument as recited in claim 1 wherein said means, connected to said second ends of said first and second main retraction portions, to move said first main retraction portion away from said second main retraction portion further comprises a scissor actuation arrangement further comprising:
a first member having a first end including a loop opening and a second end attached at an angle with respect to said first main retraction portion;
a second member having a first end including a loop opening and a second end attached at an angle with respect to said second main retraction portion, said second member pivotally connected to said first member to facilitate manual actuation of said first main retraction portion with respect to said second main retraction portion; and
means, connected between said first and said second member to selectably hold said first main retraction portion with respect to said second main retraction portion.
